(19) United States
(12) Patent Application Publication
(10) Pub. No.: US 2011/0208110 A1
(43) Pub. Date: Aug. 25, 2011

(54) Moringa oil mediated activation of the alyternative cellular energy pathway in the therapy of diseases

(76) Inventor: William John Martin, South Pasadena, CA (US)

(21) Appl. No.: 12/712,110

(22) Filed: Feb. 24, 2010

(51) Int. Cl. A61N 5/06; A61M 35/00

(52) U.S. Cl. 604/20

(57) ABSTRACT

A convenient method for activating the alternative cellular energy (ACE) pathway is described that uses ultraviolet (UV) light illumination of oil extracted from the leaves of the moringa oleifera tree. The moringa leaf oil acts as an “enerceutical,” a term defined by the inventor. The green colored moringa leaf oil can be either applied directly to areas of the skin or placed onto an impermeable barrier that is laid onto the skin. The oil displays a red fluorescence when illuminated by either mercury vapor UV emitting lamps, or bright sunlight. Smearing of the moringa leaf oil over wide surface areas of the skin in conjunction with UV illumination can be used for systemic activation of the ACE pathway. Evidence for successful systemic activation of the ACE pathway includes the appearance of direct UV light inducible tissue and body fluid fluorescence at sites other than where the oil has been applied. The induced tissue fluorescence is more yellowish-orange color, as opposed to the markedly red fluorescence of the UV light illuminated moringa leaf derived oil. As shown in other studies, systemic activation of the ACE pathway is expected to lead to clinical improvements in a wide range of infectious, metabolic and degenerative illnesses. More limited application of the UV light fluorescing moringa leaf oil to individual skin lesions can alternatively be used to specifically expedite the healing of localized skin lesions. Examples include viral infections, in which ACE pigments are known to normally accumulate.

### CROSS REFERENCE TO RELATED APPLICATIONS

Co-Pending Patent Application

Methods for Detection of Ultraviolet Light Reactive Alternative Cellular Energy Pigments (ACE-pigments) William John Martin Filed Dec. 24, 2007. Number 20090163831

Method of Assessing and of Activating the Alternative Cellular Energy (ACE) Pathway in the Therapy of Diseases. William John Martin Filed Jan. 17, 2008. Number 20090181467

Enerceutical activation of the alternative cellular energy (ACE) pathway in therapy of diseases. Filed Feb. 11, 2008 Number 20090202442

Enerceutical mediated activation of the Alternative Cellular Energy (ACE) pathway in the therapy of diseases. Filed May 8, 2008 Number 20090280193

Previously Submitted but Now Abandoned patent applications

10/044,683.Therapy of stealth virus associated cancers and other conditions using light. William John Martin. (Abandoned)

10/047,313. Therapy of stealth virus associated cancers and other conditions using medium chain triglycerides. William John Martin. (Abandoned)

10/050,232. Diagnosing and monitoring the therapy of stealth virus infections based on the detection of auto-fluorescent material in hair. William John Martin. (Abandoned)

10/058,480. Therapy of stealth virus associated cancers and other conditions using magnetic energy. William John Martin. (Abandoned)

10/164,258 Energy supportive therapy of stealth virus associated diseases. William John Martin. (Abandoned)

10/174,466 Sound therapy of stealth virus associated diseases. William John Martin. (Abandoned)

10/192,936 ACE-Pigments and humic acids as energy sources. William John Martin. (Abandoned)

Submitted: 10/______ Methods for Collection of Alternative Cellular Energy Pigments (ACE-pigments). William John Martin. (Abandoned)

Submitted: 10/______ Methods for Elimination of Toxic Alternative Cellular Energy Pigments (ACE-pigments) and for Their Replacement Using Activated Humates, including Humic and Fulvic Acids. William John Martin. (Abandoned)

United States patents (Awarded)

5,985,546 Stealth virus detection in the chronic fatigue syndrome. William John Martin

5,756,281 Stealth virus detection in the chronic fatigue syndrome. William John Martin

5,753,488 Isolated stealth viruses and related vaccines. William John Martin

5,703,221 Stealth virus nucleic acids and related methods. William John Martin

PCT (Patent Cooperation Treaty)

WO 92/20797 Stealth virus detection in the chronic fatigue syndrome. William John Martin

WO 99/34019 Stealth virus nucleic acids and related methods. William John Martin

WO 99/60101 Stealth viruses and related vaccines. William John Martin

### REFERENCES TO PUBLISHED ARTICLES

Alternative Cellular Energy Pigments (ACE-pigments):

1 Martin W J. Alternative cellular energy pigments mistaken for parasitic skin infestations. Exp. Mol. Path 78: 212-214, 2005.

2 Martin W J. Alternative cellular energy pigments from bacteria of stealth virus infected individuals. Exp. Mol. Path 78: 217-217, 2005.

3 Martin W J. Progressive Medicine. Exp Mol Path 78: 218-220, 2005.

4 Martin W J, Stoneburner J. Symptomatic relief of herpetic skin lesions utilizing an energy based approach to healing. Exp. Mol. Path 78: 131-4, 2005.

5 Martin W J. Etheric Biology. Exp Mol Path 78: 221-227, 2005.

6 Martin W J. Stealth Virus Culture Pigments: A Potential Source of Cellular Energy. Exp. Mol. Pathol. . 74: 210-223, 2003.

7 Martin W J. Complex intracellular inclusions in the brain of a child with a stealth virus encephalopathy. Exp. Mol. Pathol. 74: 179-209, 2003.

8 Martin W J. Photons and phonons: Theoretical aspects of biophysics and potential therapeutic applications. Proceeding of Neural Therapy Workshop on Sound and Light Therapy, Seattle, WA, February 21-23, 2003.

Stealth Adapted Viruses

1 Martin W J Chronic fatigue syndrome among physicians. A potential result of occupational exposure to stealth viruses. Explore 2001; 10: 7-10.

2 Martin W J. Stealth Viruses. Explore 2001; 10: 17-19.

3 Durie G M, Collins R. Martin W J. Positive stealth virus cultures in multiple myeloma. A possible explanation for neuropsychiatric co-morbidity. Presented at the Am. Soc. Hematology annual meeting October 2000.

4 Martin W J. Chemokine receptor-related genetic sequences in an African green monkey simian cytomegalovirus-derived stealth virus. Exp Mol Pathol. 2000; 69:10-6.

5 Martin W J., Anderson D. Stealth virus epidemic in the Mohave Valley: severe vacuolating encephalopathy in a child presenting with a behavioral disorder. Exp Mol Pathol. 1999; 66:19-30.

6 Martin WJ. Melanoma growth stimulatory activity (MGSA/GRO-alpha) chemokine genes incorporated into an African green monkey simian cytomegalovirus-derived stealth virus. Exp Mol Pathol. 1999; 66:15-8.

7 Martin WJ. Bacteria-related sequences in a simian cytomegalovirus-derived stealth virus culture. Exp Mol Pathol. 1999; 66:8-14.

8 Martin W J. Stealth adaptation of an African green monkey simian cytomegalovirus. Exp Mol Pathol. 1999; 66:3-7.

9 Martin W J. Cellular sequences in stealth viruses. Pathobiology 1998; 66:53-8.

10 Martin W J. Detection of RNA sequences in cultures of a stealth virus isolated from the cerebrospinal fluid of a health care worker with chronic fatigue syndrome. Case report. Pathobiology. 1997; 65:57-60.

11 Martin W J., Anderson D. Stealth virus epidemic in the Mohave Valley. I. Initial report of virus isolation. Pathobiology. 1997; 65:51-6.

12 Martin W J. Simian cytomegalovirus-related stealth virus isolated from the cerebrospinal fluid of a patient with bipolar psychosis and acute encephalopathy. Pathobiology. 1996; 64:64-6.

13 Martin W J. Stealth viral encephalopathy: report of a fatal case complicated by cerebral vasculitis. Pathobiology. 1996; 64:59-63.

14 Martin W J. Genetic instability and fragmentation of a stealth viral genome.

Pathobiology. 1996; 64:9-17.

15 Martin W J. Severe stealth virus encephalopathy following chronic-fatigue-syndrome-like illness: clinical and histopathological features. Pathobiology. 1996; 64:1-8.

16 Martin W J. Stealth virus isolated from an autistic child. J Autism Dev Disord. 1995; 25:223-4.

17 Gollard R P, Mayr A., Rice DA, Martin W J. Herpesvirus-related sequences in salivary gland tumors. J Exp Clin Cancer Res., 1996; 15: 1-4.

18 Martin W J., Glass R T. Acute encephalopathy induced in cats with a stealth virus isolated from a patient with chronic fatigue syndrome. Pathobiology. 1995; 63:115-8.

19 Martin W J, et al. African green monkey origin of the atypical cytopathic ‘stealth virus’ isolated from a patient with chronic fatigue syndrome. Clin Diag Virol 1995: 4: 93-103.

20 Martin WJ. Stealth viruses as neuropathogens. CAP Today. 1994; 8: 67-70.

21 Martin W J. et al. Cytomegalovirus-related sequence in an atypical cytopathic virus repeatedly isolated from a patient with chronic fatigue syndrome. Am J Pathol. 1994; 145: 440-51.

22 Martin W J. Activation of the alternative cellular energy (ACE) pathway as natural therapy for patients with autism. Submitted Mar. 27, 2008 to J. Autism and Developmental Disorders.

23. Martin W J. Activation of the alternative cellular energy (ACE) pathway as natural therapy for herpes simplex and herpes zoster virus infections. Submitted Mar. 27, 2008 to J. Infectious Diseases and subsequently to J. Complimentary and Alternative Medicine (Not accepted).

24. Activation of the Alternative Cellular Energy (ACE) Pathway as Natural Therapy for Patients with Autism. Submitted originally to J. autism and Developmental Disorder, and in a revised and expanded form to Proceedings of the National Academy of Sciences and next to Autism. Not accepted by these journals.

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not applicable.

### BACKGROUND OF THE INVENTION

Alternative cellular energy pigments (ACE-pigments) provide a source of cellular energy that is different from that provided through the oxidative metabolism of foods, or in the case of plants and certain bacteria, through the process of photosynthesis. Using the analogy of miniature batteries, the energy levels of ACE pigments in humans have been classified as being essentially uncharged when they will fluoresce when exposed to ultraviolet (UV) light but only if first mixed with a suitable triggering dye, such as freshly prepared neutral red. Upon partial charging, the ACE pigments will now also fluoresce when directly illuminated with a UV light, even in the absence of a triggering dye such as neutral red. When fully charged, ACE pigments will not receive additional energy input and will no longer fluoresce when UV illuminated, with or without the presence of neutral red dye. Activating the ACE pathway in humans, animals and plants can enhance normal cellular functions, including promoting the recovery from various infectious and metabolic diseases. While it is possible to directly activate a living organism's own ACE pigments, an alternative approach is to utilize similar cellular energy activating materials that can be obtained from various sources, including other living organisms and from certain formulations of herbal and non-herbal components. The term “enerceutical” was introduced to describe ACE pigment-like therapeutic products that could be used to indirectly activate the ACE pathway. By definition enerceuticals: i. Can provide therapeutic benefits to plants, animals and humans. ii. Unlike typical pharmaceuticals, are not restricted in their therapeutic usefulness to only specific diseases. iii. Do not have to actually localize to the cellular site of disease pathology since they exert their beneficial effects by radiating an energy field that can help improve overall cellular function, and in certain circumstances, can clearly lead to an indirect activation of an organism's own ACE pigments. Enerceutical products can, therefore, be used on skin or mucosal surfaces or even directly placed onto a covering that is placed onto a body surface area. Additional information on the ACE pathway and on using enerceuticals to activate this pathway is provided in the cited co-pending patent applications, which are included by reference into the present application.

Efforts have been undertaken to identify naturally occurring enerceutical products that can activate the ACE pathway in humans and animals. An initial focus has been on using such products in patients infected by stealth adapted viruses, which are not effectively targeted by the cellular immune system. Many of these patients were shown to have a deficiency in their ACE pathway by virtue of having saliva, perspiration and/or urine, which will become fluorescent after mixing with freshly prepared neutral red (at approximately 1 mg/ml) and illuminating for up to several minutes with a regular UV light. The testing can be performed either directly on fluids or on Q-tips or other types of swabs used to collect the bodily fluids. Certain ACE deficient individuals will show limited direct fluorescence of these fluids, or more commonly of the posterior oral cavity mucosa, upon direct exposure to UV light. A subset of these patients actually produce aggregated ACE pigments in the form of fluorescent aggregated particles and threads, which can be mistaken for parasites. This condition has been referred to as Morgellon's disease and is yet another example of the many manifestations of stealth adapted virus infections. Generally, however, in stealth adapted virus infected patients, the UV fluorescence of their bodily fluids is only seen after adding a suitable triggering dye such as neutral red. Demonstrable ACE deficiency also occurs in, and can contribute to, many other types of infectious, metabolic, nutritional and degenerative diseases.

Enerceutical activation of the ACE pathway can lead to a situation where the addition of neutral red dye is no longer required to visualize UV fluorescence of the skin, oral cavity or bodily fluids. This change is interpreted as indicating partial, but not yet full charging of the ACE pathway in the treated individuals. The direct fluorescence of bodily fluids and areas of untreated skin will persists for varying periods (approximately 5-30 minutes) after the treatment is discontinued. For example, video recorded observations indicate bright fluorescence occurring on the sole of a young boy and on the tongue of an adolescent girl, both of whom had just finished being treated for their autism with a UV illuminated enerceutical product. Although, the direct fluorescence gradually fades, fluorescence will still usually be seen with the addition of neutral red to a bodily fluid sample obtained after the cessation of the direct UV inducible fluorescence. Although yet to be achieved, the goal for both of these and other children with autism is to reach a point where their ACE pathway is fully activated and neutral red will no longer be able to trigger any discernable fluorescence from their bodily fluids.

The main enerceutical product tested in the two autistic children has been a Lidocaine (xylocaine) containing herbal enerceutical formulation referred to as Epione. This product was developed after testing various homeopathic formulations and reviewing their stated contents. Although denied being used by the manufacturer, Lidocaine was detected in one of the herbal preparations that was clinically effective when injected intramuscularly into children with tropical diarrhea. Although this product did not show UV fluorescence when mixed with neutral red dye, several modified formulations that comprised <1% Lidocaine and highly diluted herbal tinctures, would display striking yellow to orange fluorescence when applied with neutral red to UV illuminated paper towels. A method was developed for activating the ACE pathway in children with autism by simply laying neutral red stained paper towels onto a Saran wrap polyethylene covered area of the body, spraying Epione onto the neutral red moistened paper towels and illuminating the towels with UV light. In addition to being clinically beneficial, this procedure clearly activated the body's ACE pathway when used in children with autism and when used to locally treat skin lesions caused by herpes simplex virus (HSV), herpes zoster virus (HZV) and human papillomavirus (HPV) infections. As noted above, systemic activation of the ACE pathway was shown by direct UV inducible fluorescence becoming present within non treated areas of the skin and within the oral cavity of treated patients with autism. Herpes skin lesions would also become locally highly fluorescent even though no actual contact was made between the neutral red dye or Epione solution with the herpes skin lesions. Additional studies indicated that the energy transfer between the activated Epione enerceutical solution and the body was not simply mediated by the light of the fluorescence. Thus, visible light blocking black plastic could be used instead of Saran wrap and still elicit systemic ACE pathway activation. The current data are at least suggestive of a vibrational force that can seemingly resonate with the body's own ACE pigments.

Unfortunately, the FDA and Health Canada found objections to the parents' use of Lidocaine. Although, in my opinion, it was essentially being uses a medical device for activating the ACE pathway, the regulatory authorities insisted that the Lidociane would still be classed as a drug. As an alternative approach, I was able to confirm on repeated occasions that saliva and/or urine collected from an autistic child and from an ACE pathway deficient adult, could substitute for the Epione enerceutical solution. In a typical experiment, saliva would be collected over several hours and placed in a plastic bag with a sheet of paper to absorb the saliva. An approximately one-tenth to one half volume of freshly prepared neutral red (estimated as 1 mg/ml) in water was added to the bag and its fluorescence confirmed under UV illumination. The plastic bag would be placed over a part of the body, e.g., back, abdomen, palms of the hand and soles of the feet. UV illumination would be applied for approximately an hour. Without exception, fluorescence with UV illumination would occur elsewhere in the body and, in particular, in the oral cavity. UV illuminated saliva mixed with neutral red has also been similarly used to treat HSV and other types of localized skin lesions.

The therapeutic use of saliva and of urine mixed with neutral red has some practical limitations and continuing efforts have been made to identify alternative sources of enerceuticals. Consideration has been given to products such as humic and fulvic acids, a terpene rich extract from Japanese trees (HB-101) and others. While they have many reported properties consistent with being a source of ACE, such as promoting the growth and vitality of plants, and achieving clinical benefits in humans, they were not directly responsive to UV illumination, either in the presence or absence of neutral red dye. Further consideration was, therefore, given to natural products derived from plants with exceptional growth stimulatory activities. Included in the initial selection of plants were the Ashitaba angelica (so called tomorrow's leaf from Japan), Gynostemma pentaphyllum (longevity herb from China), and Moringa oleifera (miracle tree originally from India but now extensively cultivated in the Philippines). Clinical reports of individuals consuming these three products are consistent with their having ACE pathway activating capacity. It was repeatedly emphasized, however, that the freshly harvested leaves from these three plants were far superior in their medicinal and vitality enhancing property that were processed products, such as dried leaf powder.

Of the products so far tested, concentrated oil extracted from the leaves of the moringa tree is providing positive ACE pathway activating activity in both patients and in other plants

### BRIEF SUMMARY OF THE INVENTION

The invention describes the use of an oil extract derived from the leaves of moringa oleifera tree leaves as an enerceutical for the purpose of enhancing the body's ACE pathway in the therapy of diseases. The therapy is applicable to the treatment of a wide range of illnesses in which a deficiency of this pathway is contributing to the disease process. A primary emphasis is given to patients infected with atypical (stealth adapted) viruses that are not effectively recognized by the cellular immune system. Recovery from these infections is, therefore, more dependent upon the ACE pathway than is the recovery from most conventional viruses, for which the immune system is also operative. Activation of the ACE pathway is nevertheless potentially beneficial in all infectious diseases, as well as metabolic and degenerative illnesses. The moringa leaf derived oil is applied onto, or placed into close proximity to the skin and rendered fluorescent with UV illumination. Energy from the fluorescing oil is apparently transmissible to the body of ACE energy deficient individuals, since it commonly results in other skin areas and in the oral cavity becoming fluorescent when directly illuminated with a UV light. The method is also applicable to the therapy of localized skin lesions, such as infections, psoriasis and eczema, which can also show direct fluorescence during therapy, which is clearly distinguishable from the fluorescing moringa leaf derived oil.

### BRIEF DESCRIPTION OF THE DRAWINGS

Not Applicable and none included

### DETAILED DESCRIPTION OF THE INVENTION

Rather than relying upon neutral red to interact directly with ACE pigments derived from a patient, I have discovered that a UV light fluorescing oil derived from the leaves of the moringa oleifera tree can also be used to activate the body's ACE pathway. The oil was produced as a dietary product by Manila Nature's Link, a subsidiary of Orion Energy Incorporated in the Philippines. This green colored semi-viscous oily material was chosen from a variety of moringa products tested because it fluoresced brightly when UV illuminated and because it yielded positive clinical findings when clinically tested. The oil is packaged in 500 mg soft gel capsules, with some additional oil derived from moringa seeds. As a control, similar capsules that only contained oil derived from moringa seeds were obtained from the same manufacturer. The seed derived oil was colorless and did not fluoresce under UV illumination. Examples of clinical testing include:

A small quantity, estimated as 100 mg, of oil was smeared onto the back of a 5 year old boy with autism. The area was then illuminated with a UV light source. Bright red fluorescence was seen where the oil had been applied. Within 10-20 minutes, more yellowish deep skin fluorescence gradually developed within the surrounding skin. The parent applying the therapy had previously gained experience using Epione spray and knew well what to expect with the appearance of a directly fluorescing reaction within the skin. In a repeat study, applying the oil to the palm of one hand of the child led to the delayed but unmistakable appearance of yellowish orange fluorescence on the other palm and on the soles of the child's feet. The several therapy sessions were reported by both the mother and the father of the child as leading to improved alertness and social interaction with clearer pronunciation of words. Clinical improvement was also noted in the sibling of this child and in another child treated for eczema.

In an adult patient, the extent and intensity of UV visible yellowish oral fluorescence was quite markedly increased within minutes after the application of the leaf derived oil to the face and in another experiment, to the palms of the patient followed by UV illumination. Consistent with previous observations with other enerceuticals, the moringa oil could be spread onto the outer surface of “clinging saran™ wrap” that was laid across the palm of the patient's hand so as to avoid direct skin contact with the moringa oil. This observation is in keeping with the concept that enerceuticals can achieve their desired effect by creating an energy field that can permeate through the body. Neither direct nor evoked fluorescence could be obtained using the moringa seed oil instead of the moringa leaf oil.

UV illuminated and, therefore, red fluorescing moringa oil was also applied to a small localized wart on the index finger of a patient. While it is too early to report complete regression of the wart, exposure to the moringa oil evoked a pale yellowish fluorescence within the wart during the 30 minutes of UV light exposure. Additional treatments with moringa leaf derived oil are planned for warts, herpes simplex virus (HSV) induced lesions, herpes zoster virus (HZV) induced shingles, psoriasis and other types of skin lesions. A preliminary study has also been started for the local treatment on the gums of a subject with mild periodontal disease. Although only the lower gums were smeared with the moringa leaf oil, the resulting induced tissue fluorescence involved the upper gums, palate and back of the throat. Clinical improvement appeared to occur.

A 15 watt ProLume ultra compact mini-spiral mercury vapor black light was used for most of the preceding studies (Obtained from Halco Light Technologies, Norcross Ga.). It has a maximum emission at 365 nm. The induced skin fluorescence is best observed in a dark room by placing a UV transparent but visible light blocking filter between the UV light and the skin.

Based on beneficial effects seen on outdoor plants on which I have smeared some of the moringa leaf oil, it is reasonable to assume that the UV component in sunlight might well interact with the oil. If so, this would allow for the oil to be used in sunlit outdoor environments, without the need for a UV light.

The active components within the moringa leaf oil, which are seemingly transducing UV energy into ACE and can secondarily activate the body's own ACE pigments, remain to be characterized. It may be the physical properties of these components, rather than their chemical form that determines their effectiveness as energy transducers. The level of fluorescence seen with the moringa leaf oil is rather striking and contrasts sharply with the lack of fluorescence seen with many other natural products tested for possible enerceutical activity. These include chlorophyll rich extracts from wheatgrass, various essential oils, including frankincense and myrrh, the Japanese terpene rich product HB-101 and others. The lack of discernable fluorescence does not exclude such products as being enerceuticals, but suggests that UV light is not a suitable primary energy source for their activation.

The ease of using moringa leaf derived oil as an enerceutical makes it a suitable product for personal use. While the initial emphasis is in developing a therapy for children with autism and adults with mental illnesses, there are many other applications of locally applied enerceutical based therapies. These include HSV, HZV and HPV infections, periodontal disease, psoriasis, eczema, solar keratitis and possibly even skin malignancies. I have previously shown that cellulite can be improved using Epione and would, therefore, be expected to also benefit from moringa leaf oil. It is reasonable to also extrapolate that non-toxic ACE activating enerceuticals could be injected into lesions within the body and subsequently activated using an appropriate energy source. Certainly, UV light can be delivered via fiber optic means. Other externally applied forms of energies, such as magnetic and sound, may also be found to be capable of activating various internally delivered enerceuticals.

A deficiency in the ACE pathway will presumably place additional requirements on the oxygen consuming metabolic processing of foods as a source of cellular energy. Conversely, in individuals who are lacking in oxygen delivery from conditions such as emphysema or anemia, or who have impaired metabolic functions because of inherited or degenerative diseases, it is likely that there will be an increased reliance on the ACE pathway. By enhancing the ACE pathway, it is reasonable to expect some amelioration of the underlying symptoms of these diseases. This prediction has been validated in Epione based therapy of a patient with emphysema and similar results are expected using UV illuminated moringa lead oil. Clinical trials in diabetic and hypertensive patients are also planned.

It is also likely that the consumption of enerceutical products, including fresh and processed moringa leaves, can augment the body's internal ACE pathway by becoming an additive source of the body's ACE pigments. Such determinations can be made by assessing the level of ACE pathway activity before and after consuming the enerceutical product. Such an approach is particularly attractive as a potential means of preventing the onset of illnesses, such as autism and psychosis, and also possibly delaying ageing.

The principles, preferred embodiments and modes of operation of the present invention have been described in the foregoing specification. The invention which is intended to be protected herein, however, is not to be construed as limited to the moringa leaf oil, since this is to be regarded as illustrative of one of possibly many suitable plant derived products to be used as an enerceutical. Nor is the invention intended to be necessarily restricted to a manmade UV light as the source of physical energy. Indeed, exposure of locally applied moringa leaf oil to sunlight may suffice. Moreover, it is likely that portions of the electromagnetic spectrum other than UV light and other forms of energy such as magnetic, electrostatic and vibrational, will prove to be useful with various enerceuticals for activation of the ACE pathway in an ACE energy-deprived subject. Nor is the invention dependent upon the precise decision as to what does or does not constitute an enerceutical. Basically, the claims relate to the broader issue of achieving therapeutic benefit through activation of the ACE pathway with the description of a currently practiced method. Additional advantages and modifications will readily occur to those skilled in the art and especially upon practicing the currently described methods. Variations and changes may be made without departing from the spirit of the invention encompassed by the appended claims.

What I claim as my invention is:

1. A method for activating the alternative cellular energy (ACE) pathway in an ACE energy deficient human or animal subject, comprising the steps of applying oil derived from the leaves of a moringa tree directly to the skin and/or mucus membrane of the subject, followed by ultraviolet (UV) light illumination of the oil for sufficient time to energize the subject's ACE pathway, as shown by the development of direct UV inducible fluorescence in areas of the body, which were not directly fluorescent under direct UV light illumination prior to the therapy; and for the purpose of expediting the healing of a disease process affecting the subject.

2. A method for activating the alternative cellular energy (ACE) pathway in an ACE energy deficient human or animal subject, comprising the steps of applying oil derived from the leaves of a moringa tree onto a material that is placed onto the skin or mucus membrane of the subject, followed by ultraviolet (UV) light illumination of the oil for sufficient time to energize the subject's ACE pathway, as shown by the development of direct UV inducible fluorescence in areas of the body, which were not directly fluorescent under direct UV light illumination prior to the therapy; and for the purpose of expediting the healing of a disease process affecting the subject.

3. A method for activating the alternative cellular energy (ACE) pathway within a localized lesion on the skin or on a mucus membrane, comprising the steps of applying oil derived from the leaves of a moringa tree directly onto the lesion or onto a material that is laid onto the lesion, followed by ultraviolet (UV) light illumination of the oil for sufficient time to energize the ACE pigments within the lesion, as shown by the development of direct UV inducible fluorescence within the localized lesion that was not detectable prior to the therapy; and for the purpose of expediting the healing of a disease process affecting the subject.

4. The method of claim 1 in which the diseases causing the deficiency or inadequacy of the ACE pathway is presumptively caused by atypical (stealth adapted) viruses that lack antigenic components capable of activating an effective anti-viral cellular immune response and which are typically manifested by impaired functioning of the brain.

5. The method of claim 1 in which the disease is autism or a related illness, including childhood learning and behavioral disorders.

6. The method of claim 1 in which the diseases is characterized by the formation on the skin and hair of alternative cellular energy (ACE) pigments having the form of particles and fibers and that has been referred to as Morgellon's disease

7. The method of claim 1 in which the disease is a psychiatric or neurological illness.

8. The method of claim 1 in which the disease is the chronic fatigue syndrome or a related illness, including fibromyalgia and depression.

9. The method of claim 1 in which the disease is caused by hypoxia as occurs in illnesses such as emphysema and anemia, which can impair the body's ability to generate cellular energy through normal metabolic pathways.

10. The method of claim 1 in which the disease is caused by a metabolic disorder and/or nutritional deficiency, which can impair the body's ability to generate cellular energy through normal metabolic pathways.

11. The method of claim 4 in which the localized disease being treated is caused by herpes simplex virus (HSV), herpes zoster virus (HZV), or human papillomavirus (HPV).

12. The method of claim 4 in which the localized disease being treated is characterized by inflammation and includes psoriasis, eczema of the skin and periodontal disease of the gums.
